(19) United States
(12) Patent Application Publication
(10) Pub. No.: US 2011/0190244 A1
(43) Pub. Date: Aug. 4, 2011

(54) METHOD OF TREATMENT OF EGFR INHIBITOR TOXICITY

(75) Inventor: John R. Zalcberg, Caulfield North (AU)

(73) Assignee: Peter MacCallum Cancer Institute, Melbourne (AU)

(21) Appl. No.: 12/912,624

(22) Filed: Oct. 26, 2010

Related U.S. Application Data
(60) Provisional application No. 61/300,123, filed on Feb. 1, 2010.

(51) Int. Cl. A61K 31/37; A61K 31/4196; A61K 31/5685; A61P 39/02; A61P 17/00; A61P 27/02

(52) U.S. Cl. 514/171; 514/455; 514/383

(57) ABSTRACT

The invention provides a method of treating and/or preventing a toxicity associated with epidermal growth factor receptor (EGFR) inhibitor therapy in a subject, the method comprising administering to the subject an effective amount of a steroid sulfatase (STS) inhibitor. The toxicity may be ocular toxicity; or dermatologic toxicity, such as papulopustular rash. The EGFR inhibitor may be selected from the group consisting of: a small molecule; an antibody or derivative or fragment thereof; another agent that targets the extracellular or intracellular domain of the EGFR, such as a tyrosine kinase inhibitor selected from the group consisting of: erlotinib; gefitinib; lapatinib; and any combination thereof. The EGFR inhibitor may also be antibody selected from the group consisting of: cetuximab; panitumumab; and any combination thereof.

Preferably the STS inhibitor is selected from the group consisting of: alternative STS substrates; reversible STS inhibitors; and irreversible STS inhibitors; and any combination thereof. A preferred STS inhibitor is the irreversible nonsteroidal STS inhibitor STX64.

In some embodiments, the subject receiving EGFRI therapy has a cancer comprising cells that express wildtype k-ras and/or wildtype b-raf. In other embodiments, the cancer may be hormone-dependent. Cancers that may be treated with EGFRI therapy include colorectal cancer and non-small cell lung cancer.

### FIELD OF THE INVENTION

The subject specification relates to treating and/or preventing toxicities associated with epidermal growth factor receptor (EGFR) inhibitor therapy through the use of steroid sulfatase inhibitors, alone or in combination with aromatase inhibitors. In particular, the subject specification relates to the field of EGFR inhibitor therapy for cancer and the treatment and/or prevention of associated toxicities.

### BACKGROUND OF THE INVENTION

Cancer is the second leading cause of death after heart disease, accounting for approximately 1 in 4 deaths. It is also predicted that cancer may surpass cardiovascular diseases as the number one cause of death within 5 years. Solid tumours are responsible for most of those deaths. Although there have been significant advances in the medical treatment of certain cancers, the overall 5-year survival rate for all cancers has improved only by about 10% in the past 20 years. Cancers, or malignant tumours, metastasize and may grow rapidly in an uncontrolled manner, making treatment more difficult. Colorectal cancer is a leading cause of cancer mortality in the United States (Jemal et al 2009).

The epidermal growth factor receptor (EGFR) is often overexpressed or dysregulated in a variety of solid tumours. The epidermal growth factor receptor (EGFR) is a tyrosine kinase receptor from a larger family of ErbB receptors (ErbB 1-4 or HER 1-4) that mediate cell survival, proliferation, angiogenesis, and invasiveness, and dysregulated EGFR may be associated with uncontrolled cell growth, proliferation and angiogenesis (Mendelsohn 2002). Thus EGFR is a target for cancer therapy.

Depending on tumour stage and subgroup, inhibitors of EGFR, sometimes used alone and sometimes in combination with chemotherapy, have been shown to be associated with an increase in overall survival and/or progression-free survival in patients with colorectal, head and neck, pancreatic, and non-small cell lung cancers (NSCLC) and are under investigation in other tumour types. Classes of EGFR inhibitors (EGFRIs) include monoclonal antibodies, such as cetuximab and panitumumab, and small molecular weight tyrosine kinase inhibitors, such as erlotinib, gefitinib, and lapatinib, which is a dual EGFR and human epidermal growth factor receptor 2 [HER2] inhibitor (Castillo et al 2004). The monoclonal antibodies target the extracellular domain of EGFR and are given intravenously. The small molecule inhibitors of EGFR inhibit the ATP-binding site of the kinase domain and are given orally. These EGFR inhibitors are currently approved by the USFDA for certain types of metastatic cancer, such as breast, colon, head and neck, NSCLC, and pancreatic cancers. In addition, clinical trials are in progress for EGFR inhibitors in patients with other tumours reliant on aberrant signalling through this receptor class (Weber et al 2007).

The efficacy of EGFRI therapy using monoclonal antibodies such as cetuximab and panitumumab is confined to patients with non-mutated (wildtype) KRAS (Melosky et al 2009). K-RAS (V-Ki-ras2 Kirsten rat sarcoma viral oncogene homolog) is a GTPase, which in humans is encoded by the KRAS gene. Mutated KRAS genes are potent oncogenes that play a role in many cancers, including colorectal cancer.

EGFRI therapy is commonly associated with dermatologic reactions, particularly a papulopustular rash, which occurs in nearly all patients (Lacouture et al 2006). Papulopustular rash is the most common toxicity associated with EGFRI therapy, but other cutaneous effects, such as xerosis, pruritus, paronychia, and changes in hair growth, have also been observed. Inhibitors of EGFR are also known to have ocular side effects such as dry eye, inflammation of the lid margin (blepharitis), dysfunction of the sebaceous glands of the eyelid (meibomitis), long eyelashes (trichomegaly), corneal erosion, and inversion or eversion of the eyelid margin (entropion or ectropion) (Basti 2007), in addition to gastrointestinal and other side-effects (e.g. diarrhoea, headache).

The papulopustular rash resulting from EGFRI therapy causes chronic discomfort, itching, burning and poses a risk of secondary infections. The rash predominantly affects visible areas of the body, which can cause distress and anxiety in some patients and negatively affect self-image and self-esteem, having a negative impact on quality of life. The papulopustular rash side effect of EGFRI therapy may lead to treatment discontinuation in up to one-third of patients treated with EGFRIs (Boone et al 2007). Interestingly, however, the severity of the rash is positively correlated with the efficacy of the EGFRI therapy.

Current approaches to rash management vary widely and are based largely on anecdotal evidence and clinical experience. Accordingly, the therapeutic effect of such approaches is not clear. Such approaches include the administration of topical and oral antibiotics (e.g. tetracycline, doxycycline, minocycline, clindamycin and erythromycin), topical steroids, topical and oral retinoids (e.g. acitretin, isotretinoin), a topical vitamin K3 analog (menadione) and moisturising creams or combinations thereof.

The enzyme steroid sulfatase (STS) has been implicated in the development of acne vulgaris, which is associated with androgen excess and/or increased sensitivity to androgen. An increase in the rate of 5α-dihydrotestosterone (5α-DHT) formation from testosterone has been demonstrated in acne-prone skin compared to non-acne prone skin. In addition, acne-prone skin on the shoulder metabolises dihydroepiandrosterone (DHEA), to androgen at a greater rate than the non-acne prone skin on the chest wall or thigh. DHEA sulphate is metabolised in keratinocytes and sebaceous glands to DHEA by steroid sulphatase, and in turn is converted to 5α-DHT.

Accordingly, STS inhibitors have been used for the local treatment of acne and other androgen-dependent disorders of the pilosebaceous unit (e.g. seborrhea, androgenic alopecia and hirsutism) and also for the local treatment of squameous cell carcinoma (US Patent Application No. 2008/0293758). Additionally, STS inhibitors have therapeutic potential for the treatment of hormone-dependent cancers (Foster et al 2006). Examples of STS inhibitors include BN83495 (also known as STX64 or 667-Coumate) (U.S. Pat. No. 5,616,574) and STX213 (Fischer et al 2003).

However, therapeutic efficacy of such STS inhibitors in the treatment of papulopustular rash associated with EGFR inhibitor therapy has not previously been suggested or reported. Nor has it been reported whether there is an interaction between EGFR inhibitors and inhibitors of steroid metabolism upon co-administration.

Medications useful for the treatment of acne vulgaris (e.g. topical retinoids, benzoyl peroxide) are not indicated to treat the papulopustular rash resulting from EGFRI therapy. Patients often refer to the papulopustular rash as acne, and the term acneiform was used for initial descriptions of the rash. However, while the EGFR inhibitor-associated skin rash is often referred to as an acne-like or acneiform rash because of the inflammatory follicular appearance of the lesions (Cunningham et al 2004; Agero et al 2006; National Cancer Institute Common Terminology Criteria for Adverse Events (CTCAE) version 3.0: <http://ctep.cancer.gov/protocolDevelopment/electronic_applications/docs/ctcaev3.pdf>), it is in fact quite distinct from acne. Although the EGFR inhibitor-associated skin rash has the papules and pustules of acne vulgaris, it lacks comedones, which are the primary lesions of classic acne. Despite a similar appearance to acne vulgaris, the etiology, pathophysiology, and therapeutic approaches to EGFR inhibitor-associated exanthems are entirely different. More recently, the papulopustular rash has been described as follicular rash, folliculitis, macular/papular eruption, pustular eruption, and monomorphic pustular lesions (Agero et al 2006; Herbst et al 2002).

In view of the discussion above, there exists a need for effective treatment and/or prevention of toxicities associated with EGFRI therapy and, in particular, of papulopustular rash.

### SUMMARY OF THE INVENTION

In one aspect, the present invention provides a method of treating and/or preventing a toxicity associated with epidermal growth factor receptor (EGFR) inhibitor therapy in a subject, the method comprising administering to the subject an effective amount of a steroid sulfatase (STS) inhibitor.

An embodiment of the present invention provides a method of treating and/or preventing a toxicity associated with EGFR inhibitor therapy for a cancer in a subject, the method comprising administering to the subject an effective amount of a steroid sulfatase (STS) inhibitor.

In one embodiment of the present invention, the above methods further comprise administration to the subject an effective amount of an aromatase inhibitor.

In some embodiments, the toxicity is selected from the group consisting of: ocular toxicity; and dermatologic toxicity.

In a particular embodiment the toxicity is dermatologic toxicity.

In another embodiment, the dermatologic toxicity is papulopustular rash.

In additional embodiments of the present invention, the EGFR inhibitor is selected from the group consisting of: a small molecule; an antibody or derivative or fragment thereof; another agent that targets the extracellular or intracellular domain of the EGFR; and any combination thereof.

In some embodiments, the EGFR inhibitor targets the intracellular portion of the EGFR. In particular embodiments, such an EGFR inhibitor is a tyrosine kinase inhibitor selected from the group consisting of: erlotinib; gefitinib; lapatinib; and any combination thereof.

In other embodiments, the EGFR inhibitor targets the extracellular domains of the EGFR. In particular embodiments, such an EGFR inhibitor is an antibody selected from the group consisting of: cetuximab; panitumumab; and any combination thereof.

The STS inhibitor of the invention may be administered, alone or in combination with an aromatase inhibitor, by a route selected from the group consisting of: oral, topical; parenteral; mucosal; and any combination thereof.

In a particular embodiment of the present invention, the STS inhibitor is administered by the parenteral route, either alone or in combination with an aromatase inhibitor.

In other embodiments, the STS inhibitor and the aromatase inhibitor are administered to the subject by the same route or by different routes.

In various embodiments of the present invention, the STS inhibitor is selected from the group consisting of: alternative STS substrates; reversible STS inhibitors; and irreversible STS inhibitors; and any combination thereof.

In a particular embodiment, the STS inhibitor is the irreversible nonsteroidal STS inhibitor STX64.

In various embodiments of the invention, the aromatase inhibitor is selected from the group consisting of: anastrozole; exemestane; letrozole; and any combination thereof

In some embodiments, the subject receiving EGFRI therapy has a cancer comprising cells that express wildtype k-ras and/or wildtype b-raf.

In other embodiments, the cancer may be hormone-dependent.

The present invention provides methods suitable for use in patients with cancer selected from the group consisting of: colorectal cancer (including advanced and operable-early colorectal cancer); head and neck cancer; pancreatic cancer; non-small cell lung cancer; breast cancer; gastro-intestinal cancer; colon cancer; skin cancer; other solid tumours; leukemia and lymphoma.

Another aspect of the present invention provides a use of a STS inhibitor, alone or in combination with an aromatase inhibitor, in the manufacture of a medicament for the treatment and/or prevention of a toxicity associated with EGFR inhibitor therapy in a subject.

In an additional aspect, the present invention provides a use of a STS inhibitor, alone or in combination with an aromatase inhibitor, for the treatment and/or prevention of a toxicity associated with EGFR inhibitor therapy in a subject.

In another aspect, the present invention provides a pharmaceutical composition comprising an STS inhibitor for the treatment and/or prevention of a toxicity associated with EGFR inhibitor therapy in a subject, and a pharmaceutically-acceptable carrier.

In an embodiment of the present invention, such a composition further comprises an EGFR inhibitor and/or an aromatase inhibitor.

In a further aspect, the present invention provides a kit comprising an STS inhibitor and a pharmaceutically-acceptable carrier for the treatment and/or prevention of a toxicity associated with EGFR inhibitor therapy in a subject.

In a particular embodiment, the present invention provides a kit comprising an STS inhibitor and a pharmaceutically-acceptable carrier when used in the treatment and/or prevention of a toxicity associated with EGFR inhibitor therapy in a subject.

In further embodiments of the present invention, such kits as described above further comprise an EGFR inhibitor and/or an aromatase inhibitor.

One aspect of the present invention provides a method of treatment of cancer comprising administering to a subject in need thereof a therapeutically effective amount of an EGFR inhibitor and a therapeutically effective amount of a STS inhibitor.

In one embodiment of the present invention, the above method further comprises administration of an aromatase inhibitor to the subject.

Some embodiments of the present invention provide such a method wherein the combination of the EGFR inhibitor and the STS inhibitor, alone or in combination with an aromatase inhibitor, substantially reduces the severity of a toxicity associated with EGFR inhibitor therapy, for example, papulopustular rash, and/or increases the efficacy of the EGFR inhibitor treatment.

The above summary is not and should not be seen in any way as an exhaustive recitation of all embodiments of the present invention.

### DETAILED DESCRIPTION OF THE INVENTION

Bibliographic details of references in the subject specification are listed at the end of the specification.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

The terminology used herein is for the purpose of describing particular embodiments only and is not intended to be limiting.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

As used herein the singular forms “a”, “an” and “the” include plural aspects unless the context clearly dictates otherwise. Thus, for example, reference to “a cell” includes a single cell, as well as two or more cells; reference to “an agent” includes one agent, as well as two or more agents; and so forth.

Each embodiment in this specification is to be applied mutatis mutandis to every other embodiment unless expressly stated otherwise.

EGFR inhibitor (EGFRI) therapy is an effective treatment for cancer, in particular in some groups of patients with colorectal, head and neck, pancreatic, and non-small cell lung cancers (NSCLC). EGFRI therapy has been approved for marketing by the FDA and in some cases by the EMEA. EGFRI therapy is associated with different adverse events than conventional chemotherapy (and indeed may be used in combination with chemotherapy or radiotherapy), however, toxicities associated with EGFRI therapy, such as papulopustular rash, can be dose-limiting.

The present invention provides methods of treating and/or preventing a toxicity associated with epidermal growth factor receptor (EGFR) inhibitor therapy in a subject, the method comprising administering to the subject an effective amount of a steroid sulfatase (STS) inhibitor. A distinct advantage of treating and/or preventing toxicities associated with EGFRI therapy is that EGFRI dose modification (for example, increasing the dose of EGFRI for a period of time or continuing therapy in a patient who is benefitting clinically) is tolerable by the patient, resulting in more effective EGFRI therapy. Additionally, alleviation of toxicities such as papulopustular rash leads to an improved quality of life for the cancer patient.

One aspect of the present invention provides a method of treating and/or preventing a toxicity associated with epidermal growth factor receptor (EGFR) inhibitor therapy for a cancer in a subject, the method comprising administering to the subject an effective amount of a steroid sulfatase (STS) inhibitor, either alone or in combination with an effective amount of an aromatase inhibitor.

Reference to “EGFRI” should be understood as reference to any molecule that inhibits or down-regulates the biological activity of the EGFR. EGFRIs include, but are not limited to, molecules that fall into the following two broad categories: antibodies, which target the extracellular region of the EGFR; and small molecule inhibitors, for example tyrosine kinase inhibitors.

Examples of currently approved/marketed antibodies that are useful as EGFRIs are the monoclonal antibodies cetuximab (“Erbitux”®—BMS and MerckSerono) and panitumumab (“Vectibix”®—Amgen). Other antibodies in development for use as EGFRIs include: zalutumumab; nimotuzumab; and matuzumab.

Tyrosine kinase inhibitors (TKI' s) include: erlotinib (“Tarceva”®—Roche); gefitinib (“Iressa”®—Astrazeneca); lapatinib; axitinib; bosutinib; cediranib; dasatinib; imatinib; lestaurtinib; nilotinib; semaxanib; sunitinib; toceranib; vandetanib; and vatalanib. A number of TKI's, for example, erlotinib, gefitinib and lapatinib, are known to target the intracellular region of the EGFR.

Aromatase inhibitors (AIs) inhibit the cytochrome P-450 component of the aromatase enzyme complex responsible for the final step of estrogen biosynthesis in peripheral tissues. These drugs can be classified into first generation agents (e.g., aminoglutethimide), second-generation agents (e.g., formestane and fadrazole), and third-generation agents (e.g.,anastrozole, letrozole, and exemestane).

AIs can also be divided into type I and type II inhibitors. Type I inhibitors (e.g. exemestane) have a steroidal structure similar to androgens and inactivate the enzyme irreversibly by blocking the substrate-binding site, and are therefore known as aromatase inactivators. Type II inhibitors (e.g. anastrozole, letrozole) are nonsteroidal and their action is reversible. Anastrazole (Arimidex®), exemestane (Aromasin®) and letrozole (Femara®) are approved for clinical use by the U.S. Food and Drug Administration.

The term “antibody” is used herein in the broadest sense and specifically covers intact monoclonal antibodies, polyclonal antibodies, multispecific antibodies (e.g. bispecific antibodies) formed from at least two intact antibodies, and antibody fragments. “Antibody fragments” comprise only a portion of an intact antibody, generally including an antigen binding site of the intact antibody and thus retaining the ability to bind antigen. The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed usually against a single antigen.

The administration of EGFRIs to cancer patients is associated with toxicities, in particular cutaneous or dermatologic toxicities, and ocular toxicities. Reference herein to “toxicity” should be understood as reference to a toxic effect in a subject caused by administration of an EGFRI to the subject. The term “toxicity” is used interchangeably herein with the terms “adverse event” and “side-effect”. Reference to the terms “cutaneous” and “dermatologic” should be understood as pertaining to the skin. Reference to the term “ocular” should be understood as pertaining to the eye.

Papulopustular rash is the most common toxicity associated with EGFRI therapy, however the present invention contemplates the treatment of other dermatologic toxicities known to be associated with EGFRI therapy including but not limited to xerosis and pruritus. EGFRI therapy can also cause nail abnormalities such as paronychia, and hair growth abnormalities such as scalp and body alopecia, increased hair growth, hair curling, hypertrichosis and hyperpigmentation. Inhibitors of EGFR are also known to have ocular side effects. Accordingly, the present invention also relates to the treatment of ocular toxicities associated with EGFRI therapy including, but not limited to: dry eye; inflammation of the lid margin (blepharitis); dysfunction of the sebaceous glands of the eyelid (meibomitis); long eyelashes (trichomegaly); corneal erosion; and inversion or eversion of the eyelid margin (entropion or ectropion).

The term “papulopustular rash” used herein refers to a condition that is distinct from acne vulgaris. The EGFR inhibitor-associated skin rash is a papulopustular rash distinct from acne, but is often referred to as an acne-like or acneiform rash because of the inflammatory follicular appearance of the lesions (Cunningham et al 2004; Agero et al 2006; National Cancer Institute Common Terminology Criteria for Adverse Events (CTCAE) version 3.0: <http://ctep.cancer.gov/protocolDevelopment/electronic_applications/docs/ctcaev3.pdf>). Although the EGFR inhibitor-associated skin rash has the papules and pustules of acne vulgaris, it lacks comedones, which are the primary lesions of classic acne. Despite a similar appearance to acne vulgaris, the etiology, pathophysiology, and therapeutic approaches to EGFR inhibitor-associated dermatologic toxicities are entirely different (Burtness et al 2009). The rash has also been described as follicular rash, folliculitis, macular/papular eruption, pustular eruption, and monomorphic pustular lesions (Agero et al 2006; Herbst et al 2002).

EGFRIs are used for the treatment of cancers that comprise cells having dysregulated expression or overexpression of EGFR. EGFR is often overexpressed or dysregulated in a variety of solid tumours, including gastro-intestinal malignancies. The terms “cancer”, “cancerous”, or “malignant” refer to or describe the physiological condition in a subject that is typically characterized by unregulated cell growth. Examples of cancer include but are not limited to, carcinoma, blastoma, and sarcoma. More particular examples of such cancers include colorectal cancer, pancreatic cancer, head and neck cancer, non-small cell lung cancer, breast cancer and skin cancer. The term “cancer” is used interchangeably herein with the terms “malignancy”, “malignant tumour”, “tumour” and “neoplasm”, notwithstanding the fact that tumours and neoplasms may be malignant or benign, and thus, in the case of the latter, not cancer.

In respect of EGFRI-associated toxicities, the term “treatment” refers to an approach for managing an EGFRI-associated toxicity that has already manifested in a patient. In terms of “prevention” or “prophylaxis”, these terms refer to an approach to completely or partially prevent the onset or progression of an EGFRI-associated toxicity. In the present invention, preventitive or prophylactic approaches generally involve administration of an STS inhibitor before the onset of an EGFRI-associated toxicity in a patient and may even precede the administration of the EGFRI.

The term “solid cancer” refers to a malignancy that forms a discrete tumour mass, for example: colorectal cancer, pancreatic cancer, head and neck cancer, non-small cell lung cancer, breast cancer, and melanoma. Solid cancers contrast with, for example, lymphoproliferative malignancies such as leukemia, which may diffusely infiltrate a tissue without forming a mass.

In some embodiments, the present invention provides a method for the treatment of EGFRI-associated toxicities in patients having a cancer that has a wildtype KRAS genotype and/or a wildtype BRAF genotype.

K-RAS (V-Ki-ras2 Kirsten rat sarcoma viral oncogene homolog) is a GTPase, which in humans is encoded by the KRAS gene. Mutated KRAS genes are potent oncogenes that play a role in many cancers, including colorectal cancer.

B-RAF (V-raf murine sarcoma viral oncogene homolog B1) is a cell signalling protein involved in cell growth. BRAF is encoded by the BRAF gene, which is mutated in many types of cancer, contributing to dysregulated cell proliferation and metastatic processes.

The KRAS and BRAF genotypes can be determined by routine methods well known to a person skilled in the art, for example, the polymerase chain reaction using appropriate primers.

Colorectal cancer, commonly called bowel cancer, is generally thought to be preceded by adenomas (polyps), which if undetected become invasive cancer. “Advanced colorectal cancer” can be defined as colorectal cancer that at presentation or recurrence is either metastatic or so locally advanced that the likelihood of surgical resection improving a patient's prognosis is improbable.

“Head and neck” cancers occur inside, for example, the sinuses, nose, mouth and salivary glands and throat. These cancers are grouped together because the treatments for these cancers are often the same. Non small cell lung cancers are broadly divided by their main cell type into squamous cell, adenocarcinoma and large cell and are distinguished from small cell tumours of the lung.

The methods, compositions, uses and kits of the present invention may also be of particular use in the treatment of hormone-dependent cancers of, for example, the breast, endometrium, ovary and prostate.

Steroid sulfatase (STS) is the enzyme responsible for the hydrolysis of steroidsulfates, for example, dehydroepiandrosterone sulfate (DHEAS), to their unconjugated, biologically active forms. DHEAS is secreted in large amounts (up to 20 mg per day) by the adrenal cortex and is present in the keratinocytes and sebaceous glands of the skin. DHEAS is hydrolysed to DHEA by steroid sulphatase in keratinocytes and sebaceous glands, and in turn is converted to 5α-dihydrotestosterone (5α-DHT). An increased rate of 5α-DHT formation from testosterone has been demonstrated in acne-prone skin compared to non-acne prone skin. In addition, acne-prone skin on the shoulder metabolises dihydroepiandrosterone (DHEA), to androgen at a greater rate than the non-acne prone skin on the chest wall or thigh. Accordingly inhibitors of STS have been contemplated for the treatment of acne vulgaris.

STS is also known as EC 3.1.6.2, steryl sulfatase, arylsulfatase; sterol sulfatase; dehydroepiandrosterone sulfate sulfatase; arylsulfatase C; steroid 3-sulfatase; steroid sulfate sulfohydrolase; dehydroepiandrosterone sulfatase; pregnenolone sulfatase; phenolic steroid sulfatase; 3-β-hydroxysteroid sulfate sulfatase (International Union Of Biochemistry And Molecular Biology <http://www.chem.qmul.ac.uk/iubmb/enzyme/EC3/1/6/2.html>).

Several potent irreversible STS inhibitors have been identified, such as those described in International Patent Application No. PCT/GB92/01587, U.S. Pat. No. 5,616,574, US 2009/0182000 A1, and Reed et al 2005. All such inhibitors have as their active pharmacophore an aryl ring to which a sulfamate ester is attached. The majority of STS inhibitors are irreversible however, other classes of STS inhibitor are alternative substrates containing at least one sulphate group, and reversible inhibitors. The irreversible STS inhibitors can be classed as either irreversible steroidal STS inhibitors or irreversible nonsteroidal STS inhibitors.

An example of a nonsteroidal irreversible STS inhibitor is STX64, also known as BN83495 or 667-coumate, is a first-generation STS inhibitor (U.S. Pat. No. 5,616,574; Purohit et al., 2000). The structure of STX64 is shown below.

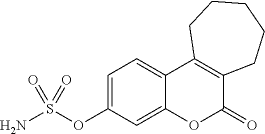

STX64 has been shown to be a potent STS inhibitor in rodents and blocked the ability of estrone sulphate (E1S) to stimulate the growth of carcinogen-induced mammary tumours in ovariectomized rats (Purohit et al., 2000). The chemical name of STX64 is:

Sulphamic acid, 6,7,8,9,10,11-hexahydro-6-oxobenzo[b]cyclohepta[d]pyran-3-yl ester

Second-generation irreversible STS inhibitors, such as the steroidal STS inhibitor STX213 have been shown to effectively inhibit STS activity in rodents (Fischer et al., 2003; Foster et al., 2006). The structure of STX213 is shown below.

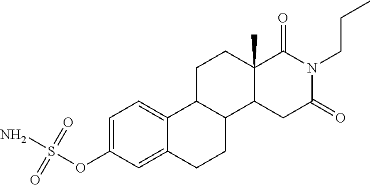

These STS inhibitors are orally active with a high level of bioavailability. However, in accordance with the methods of the present invention, the route of administration of STS inhibitors is not limited to the oral route. Other routes of administration are contemplated, for example, parenteral, topical, intravenous and mucosal.

Additional STS inhibitors useful in accordance with the present invention include SR 16157, KW-2581, Boro-001, Boro-002, Adamant-001, Adamant-002, Steroid-001, Steroid-002, Keto-001, Coumarin-001.

Aromatase inhibitors are contemplated for use in accordance with certain embodiments of the present invention and act by inihibiting the cytochrome P-450 component of the aromatase enzyme complex responsible for the final step of estrogen biosynthesis in peripheral tissues.

Examples of aromatase inhibitors (AIs) particularly useful in the present invention include anastrazole (Arimidex®), exemestane (Aromasin®) and letrozole (Femara®).

Reference to “inhibitor” herein should be understood as reference to a molecule that completely or partially inhibits the biological activity of a target molecule, for example EGFR in the case of EGFR inhibitors, STS in the case of STS inhibitors and aromatase in the case of AIs. In alternate embodiments of the invention, the percent inhibition of STS or aromatase is 100%, at least 99%, at least 98%, at least 97%, at least 96%, at least 95%, at least 94%, at least 93%, at least 92%, at least 91%, at least 90%, at least 85%, at least 80%, at least 75%, at least 70%, at least 65%, at least 60%, at least 55%, at least 50%, at least 40%, at least 30%, at least 20%, at least 10% or at least 5%. STS can be measured in any suitable tissue or cell, for example, tumour tissue, skin and peripheral blood leukocytes. STS can be found in the liver, testis, ovary, adrenal glands, placenta, prostate, skin, brain, fetal lung, viscera, endometrium, peripheral blood lymphocytes, aorta, kidney, osteoblast cells, thrombocytes and bone (Reed et al 2005). It is believed to be virtually ubiquitous in small quantities.

STS can be detected in various tissues by, for example: immunohistochemistry; biochemical analysis of hydrolytic products of various sulfated substrates by colorimetric, fluorimetric, or radiometric methods; and by determining mRNA expression levels using reverse-transcriptase PCR and/or real time PCR (Reed et al 2005).

Aromatase can be detected in various peripheral tissues by methods known by those skilled in the art.

In determining the percent inhibition of STS or aromatase, measurements of STS or aromatase made after inhibitor administration are compared to measurements made in the same patient before inhibitor administration, or are compared to the appropriate normal range.

The terms “inhibitor”, “drug”, “composition”, “agent”, “medicament” and “active” are used interchangeably herein to refer to a chemical compound or biological molecule or cellular composition which induces a desired pharmacological and/or physiological effect. The terms encompass pharmaceutically acceptable and pharmacologically active ingredients including but not limited to salts, esters, amides, pro-drugs, active metabolites, analogs and the like. The term includes genetic and proteinaceous or lipid molecules or analogs thereof as well as cellular compositions as previously mentioned. The instant compounds and compositions are suitable for the manufacture of a medicament for the treatment and/or prevention of toxicities associated with EGFRI therapy, as described herein.

Another aspect of the present invention provides a method for the combined treatment of an EGFRI-responsive cancer and a toxicity associated with EGFRI therapy, comprising administering to a subject in need thereof a therapeutically effective amount of an EGFR inhibitor and a therapeutically effective amount of a STS inhibitor, either alone or in combination with an AI.

In some embodiments, the EGFRI and the STS inhibitor are administered to a subject by different routes and/or at different times.

In those embodiments, where an AI is administered to a subject in addition to a STS inhibitor, the AI may be administered by the same route or by a different route to that used to administer the STS inhibitor.

In additional embodiments, the STS inhibitor and AI are administered to a subject at the same time or at different times.

In other embodiments, the present invention provides pharmaceutical compositions comprising a STS inhibitor and an EGFRI for co-administration to a patient via the same administration route.

In an additional embodiment, the pharmaceutical composition further comprises an aromatase inhibitor.

In some embodiments of the present invention, the EGFRI and STS inhibitor, alone or together with an AI, combine to produce a synergistic effect resulting in a more effective cancer therapy.

Any subject who could benefit from the present methods, compositions, uses or kits is encompassed herein. The term “subject” includes, without limitation, humans and non-human primates, livestock animals, companion animals, laboratory test animals, captive wild animals, reptiles and amphibians, fish, birds and any other organism. The most preferred subject of the present invention is a human subject. A subject, regardless of whether it is a human or non-human organism may be referred to as a patient, individual, subject, animal, host or recipient.

In some embodiments the present invention provides pharmaceutical compositions comprising an STS inhibitor. In additional embodiments, such pharmaceutical compositions further comprise an AI. Such compositions are useful for the treatment and/or prevention of toxicities associated with EGFRI therapy in cancer patients.

The pharmaceutical compositions of the present invention can be prepared according to conventional pharmaceutical compounding techniques. See, for example, Remington's Pharmaceutical Sciences, 20th ed. Williams and Wilkins, 2000. The composition may contain the active agent(s) or pharmaceutically acceptable salts of the active agent(s). These compositions may comprise, in addition to one of the active substances, a pharmaceutically acceptable excipient, carrier, buffer, stabilizer or other materials well known in the art. Such materials should be non-toxic and should not interfere with the efficacy of the active ingredient. The carrier may take a wide variety of forms depending on the route of administration.

Contemplated routes of administration of pharmaceutical compositions of the present invention include but are not limited to oral, parenteral, topical and mucosal (for example, intra-nasal).

Compositions of the present invention suitable for oral administration may be presented as discrete units such as capsules, sachets or tablets each containing a predetermined amount of the active ingredient; as a powder or granules; as a solution or a suspension in an aqueous or non-aqueous liquid; or as an oil-in-water liquid emulsion or a water-in-oil liquid emulsion. The active ingredient may also be presented as a bolus, electuary or paste.

A tablet may be made by compression or moulding, optionally with one or more accessory ingredients. Compressed tablets may be prepared by compressing in a suitable machine the active ingredient in a free-flowing form such as a powder or as granules, optionally mixed with a binder (e.g. cross-linked povidone, cross-linked sodium carboxymethyl cellulose), inert diluent, preservative, disintegrant (e.g. sodium starch glycollate), surface-active agent and/or dispersing agent. Moulded tablets may be made by moulding in a suitable machine a mixture of the powdered compound moistened with an inert liquid diluent. The tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Compositions suitable for topical administration in the mouth include lozenges comprising the active ingredient in a flavoured basis, usually sucrose and acacia or tragacanth gum; pastilles comprising the active ingredient in an inert basis such as gelatin and glycerin, or sucrose and acacia gum; and mouthwashes or sprays comprising the active ingredient in a suitable liquid carrier.

For topical application to the skin, the active ingredient may be in the form of a cream, ointment, jelly, solution or suspension.

Topical administration of the STS inhibitors is preferred.

Oral administration of the AIs is preferred.

For topical application to the eye, the active ingredient may be in the form of a solution or suspension in a suitable sterile aqueous or non-aqueous vehicle. Additives, for instance buffers, preservatives including bactericidal and fungicidal agents, such as phenyl mercuric acetate or nitrate, benzalkonium chloride or chlorohexidine and thickening agents such as hypromellose may also be included.

Nasal compositions may be presented topically as nose drops or sprays or systemically in a form suitable for absorption through the nasal mucosa and/or the alveolar cells in the lungs.

Compositions suitable for parenteral administration include aqueous and non-aqueous isotonic sterile injection solutions which may contain anti-oxidants, buffers, bacteriostats and solutes which render the composition isotonic with the blood of the intended subject; and aqueous and non-aqueous sterile suspensions which may include suspending agents and thickening agents. The compositions may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

The compositions of the present invention are preferably administered in a therapeutically effective amount. The actual amount administered and the rate and time-course of administration will depend on the nature and severity of the EGFRI toxicity being treated. Prescription of treatment, e.g. decisions on dosage, timing, etc. is within the responsibility of clinicians and typically takes account of the nature and severity of the EGFRI toxicity to be treated, the general condition, sex and weight of the individual patient, the route of administration and other factors known to practitioners. Examples of techniques and protocols can be found in Remington's Pharmaceutical Sciences, 20th ed. Williams and Wilkins, 2000.

The term “therapeutically effective amount” refers to an amount of a drug effective to treat a disease or condition in a subject. In the case of papulopustular rash associated with EGFRI therapy, the therapeutically effective amount of the STS inhibitor, alone or combined with an aromatase inhibitor, may reduce the frequency or incidence of the rash, reduce the severity or grade of the rash, reduce the frequency or severity of other EGFRI associated toxicities, alter estrogen and androgen related blood parameters, reduce the number of cancer cells, reduce tumour size or tumour growth, or reduce any one or more of the symptoms associated with papulopustular rash. Therapeutically effective amounts of an STS inhibitor may also improve a patient's quality of life. For example, daily dosages of from about 1 mg to about 500 mg may be appropriate, such as from about 5 mg to 250 mg per day, from about 10 mg to about 200 mg, from about 20 to about 100 mg, from about 30 mg to about 50 mg or about 35 mg, 40 mg or 45 mg per day. Such doses may for example be administered once daily or may be given in smaller twice, three or four time daily dosages. In a preferred embodiment a single daily dosage is administered after fasting, such as at least 10 min, 15 min, 20 min, 30 min or 1 hour prior to taking food (e.g. prior to breakfast). It will be well within the capabilities of a skilled medical practitioner to determine the appropriate dosage for an individual patient in view of the patent's size, age, sex, weight, general health, disease progression and previous or current experience of side effects, for example. In a particularly preferred embodiment a doage of 40 mg is administered once daily, preferably after fasting and at least 30 min prior to taking food.

In the case of cancer, the therapeutically effective amount of the drug may reduce the number of cancer cells; reduce the tumour size; inhibit (i.e., slow to some extent and preferably stop) cancer cell infiltration into peripheral organs; inhibit (i.e., slow to some extent and preferably stop) tumour metastasis; inhibit, to some extent, tumour growth; and/or relieve to some extent one or more of the symptoms associated with the disorder. To the extent the drug may prevent growth and/or kill existing cancer cells, it may be cytostatic and/or cytotoxic. For cancer therapy, efficacy in vivo can, for example, be measured by assessing the duration of survival, time to disease progression (TTP), the response rates (RR), duration of response, and/or quality of life.

The present invention also provides a use of a STS inhibitor, alone or in combination with an aromatase inhibitor, in the manufacture of a medicament for the treatment and/or prevention of a toxicity associated with EGFR inhibitor therapy in a subject.

In another aspect, the present invention provides a use of a STS inhibitor, alone or in combination with an aromatase inhibitor, for the treatment and/or prevention of a toxicity associated with EGFR inhibitor therapy in a subject.

Another aspect of the present invention provides a kit comprising an STS inhibitor and a pharmaceutically-acceptable carrier when used in the treatment and/or prevention of a toxicity associated with EGFR inhibitor therapy in a subject.

In one embodiment, the kit further comprises an EGFR inhibitor and/or an aromatase inhibitor.

The present invention is further described by the following non-limiting Examples.

### EXAMPLE 1

### The Effect of STX64 on Papulopustular Rash Associated With EGFR Inhibitor Therapy for Advanced Colorectal Cancer

The effect of administration of STS inhibitors on papulopustular rash in cancer patients undergoing treatment with EGFR inhibitors can be assessed in a randomised, placebo-controlled Phase 2 Study of the STS inhibitor, STX64 (also known as BN83495 or 667-coumate), in patients receiving an EGFR inhibitor for the treatment of advanced colorectal cancer.

Study Design

Patients with advanced colorectal cancer receiving single agent cetuximab or panitumimab are randomised to STX64 or placebo in a double-blind randomised phase 2 study.

Eligibility/Exclusion Criteria
a) Advanced colorectal cancer able to receive cetuximab or panitumimab.
b) Tumour genotype is k-ras and/or B-raf wild-type.
c) Acceptable biomedical and haematological baseline values.
d) Measureable disease.
e) Not receiving other hormonally active agents.
f) No history of prior breast, prostate, endometrial or ovarian cancer.

Study Endpoints
a) Frequency/grade of papulopustular rash.
b) Frequency of additional cutaneous (side) effects.
c) Tumour response rate.
d) Side effect profile other than skin.
e) Quality of life.
f) Changes in estrogen and androgen related blood parameters.

Treatment Duration

Treatment with STX64 is continued as long as cetuximab or panitumimab is continued, unless adverse effects thought to be due to STX64 are noted, in which case both the placebo and STX64 are discontinued without unblinding the study.

Statistics

Patients are randomised in order that the experimental arm is powered to show a 75% reduction in grade 3 rash and a 50% reduction in grade 2 rash. Analysis is conducted at 4 weeks, 6 weeks and 12 weeks after commencement of therapy because the papulopustular rash occurs in most patients within 4 to 6 weeks of starting cetuximab.

### EXAMPLE 2

Grading Papulopustular Rash

The severity of the papulopustular rash can be graded using the National Cancer Institute's (NCI) common terminology criteria for adverse events (CTCAE) (Gridelli et al 2008; CTCAE version 3.0: <http://ctep.cancer.gov/protocolDevelopment/electronic_applications/docs/ctcaev3.pdf>) Adverse events are graded on a scale of 1 to 5, with grade 1 being the mildest and least symptomatic. A grade 3 papulopustular rash is defined as a “symptomatic and disfiguring” rash. The rash typically occurs on a limited extent of skin but may be dose-limiting, with respect to EGFR inhibitors, when painful, confluent, or superinfected. The NCI's CTCAE can also be used to grade other dermatologic problems that may occur with EGFR inhibitors (e.g., hair loss/alopecia, dry skin, nail changes, pruritus/itching, telangiectasias).

### EXAMPLE 3

### The Effect of STX64 (BN83495) on Papulopustular Rash Associated With EGFR Inhibitor Therapy for Non-Small Cell Lung Cancer

The effect of administration of STS inhibitors on papulopustular rash in cancer patients undergoing treatment with an oral EGFR inhibitor can be assessed in a pilot study involving administration of the EGFR inhibitor (erlotinib or gefitinib) and BN83495, in patients receiving the EGFR inhibitor for the treatment of non-small cell lung cancer (NSCLC).

The study will enable determination of the frequency and grade of papulopustular rash in NSCLC patients receiving the the EGFR inhibitor and BN83495; the frequency and grade of additional cutaneous side effects and the changes in oestrogen and androgen-related biochemistry and EGFR inhibitor blood levels associated with the treatment.

Pharmacokinetics

In non-clinical studies BN83495 showed rapid absorption and good oral bioavailability (60 to 96% in rats and 77 to 90% in dogs). In 6-month repeat dose rat toxicology studies, plasma exposure on Day 1 was proportional to dose and there were no gender effects. However, there was a slightly lower exposure over time in rats compared to dogs. BN83495 is sequestered in erythrocytes, the uptake and retention being related to carbonic anhydrase II enzyme. BN83495 is widely distributed in rats primarily in blood and related organs, intestine, liver and kidneys. Both BN83495 and its metabolite IDP-17619 are highly protein bound. In vitro and in vivo metabolic studies have shown a large number of metabolites with qualitatively similar in vitro profiles in rats, dogs and man. Elimination of radioactivity in preclinical models was by biotransformation and was mainly via urine, although enterohepatic recirculation was noted in the rat. The only potential clinically significant induction/inhibition of a CYP450 isoenzyme by BN83495 is inhibition of CYP2C19, which is not affected by EGFR inhibitors. Based on the pharmacokinetic parameters of BN83495, it is not expected to have any significant interaction with EGFR inhibitor/s which are metabolised predominantly by CYP3A4 (Roche products—Tarceva Product Information)

Toxicology

Safety pharmacology studies performed in rats showed no effect on behaviour or respiration following single oral doses up to 1200 mg/kg. When tested in vitro, BN83495 and IDP-17619 were shown to inhibit hERG current (IC50 values of 13.0 μM and 18.3 μM, respectively) in transfected HEK-293 cells. BN83495 also increased action potential duration (APD) in isolated Purkinje fibres, but only at a high concentration of 50 μM. Oral administration of BN83495 to radiotelemetry instrumented dogs at doses of 150 and 400 mg/kg/day for seven consecutive days did not induce any clinically relevant ECG changes. The mean maximum plasma exposure at the highest tested dose was found to correspond to levels which were 62- and 103-fold the mean steady state plasma AUC and Cmax in female patients (1293 ng.h/mL and 159 ng/mL, respectively) of female patients administered an 80 mg oral dose in the current Phase I clinical study (X-55-58064-002). However, in the latter dog study, there was a transient hypotension and reflex tachycardia at both doses.

Single dose oral toxicity studies did not show any adverse effects of BN83495 in mice and rats at 2000 mg/kg. Intravenous LD50 values were 15 to 30 mg/kg in mice and 50 to 70 mg/kg in rats. Repeated dose 1- and 6-month oral toxicity studies in rats and dogs showed that the rat is the most sensitive species. In rats administered doses of 25, 50, 100 and 200 mg/kg/day, the liver showed reversible hepatocellular vacuolation in all treated groups, together with hepatocellular hypertrophy for some high dose animals. The femur, tibia and sternum showed partially reversible osteoid deposition at all treated dose-levels and increased trabecular bone from 50/100 mg/kg/day in females/males. The no observed adverse effect level (NOAEL) was 200 mg/kg/day for males and 50 mg/kg/day for females. The exposure (AUC) was 14-fold (female) and 31-fold (male) higher in rats than the mean AUC in female patients following an 80 mg oral dose. In dogs administered doses of 50, 250 and 1000 mg/kg/day, the liver presented with hepatocellular vacuolation in both sexes at 50 mg/kg/day and in females at 250 and 1000 mg/kg/day. The kidney had flattened tubular epithelium in one high dose animal/sex, whilst adrenal cortical cell hypertrophy was seen in all treated animal groups. All findings showed either full or partial reversibility.

Clinical Data

The clinical efficacy and safety of BN83495 was investigated in a Phase I study of 14 postmenopausal women with locally advanced, or metastatic, oestrogen receptor or progesterone positive, breast cancer. All of these heavily treated metastatic patients had received endocrine therapy before study entry. The patients received three 14-day cycles of BN83495 at doses of 5 mg or 20 mg administered daily on Days 1 to 5 followed by 9 days without treatment. The best response was stable disease in 4 patients (one received the 5 mg dose and three received the 20 mg dose of BN83495). At 24 hours after dosing on Day 5 the STS activity in peripheral blood lymphocytes was inhibited by more than 90% in both dose groups. This effect was also seen in tumour tissue in 3 out of 4 patients at 5 mg and in all 3 patients in whom it was measured at 20 mg. Inhibition of hydrolysis of DHEAS by BN83495 resulted in significant decreases in serum DHEA concentration. There were also significant decreases in serum concentrations of oestradiol, androstenediol and testosterone at the Day 5 assessment. BN83495 was well tolerated at both tested doses. Pharmacokinetic data indicated that BN83495 was eliminated slowly from the plasma (half-life 28 to 31 hours). Cmax, AUC and mean plasma clearance showed non dose proportional pharmacokinetics.

In 2007, a Phase I dose escalation study was initiated to determine the optimal biological dose of BN83495, after single and repeated administration, in 35 postmenopausal women with oestrogen-receptor positive breast cancer whose disease progressed after prior therapy for locally advanced/metastatic disease. This ongoing study (last patient last visit is planned Q2 2010 after the current amendment), is intended to assess the optimal biological/recommended dose, based on the safety and the biological activity of doses ranging from 1 mg up to 80 mg. It is also intended to characterise any observed clinical and metabolic drug activity at the determined recommended dose. The available information indicates that BN83495 is well tolerated up to a dose of 40 mg and no dose limiting toxicity was observed. 40mg was determined to be the optimal biological dose based on an almost complete (95%) inhibition of the target enzyme STS in peripheral blood mononuclear cells at this level. Only 4 drug-related adverse events (fatigue Grade 3 [n=2], anorexia Grade 3, nausea Grade 3 [n=1] and vomiting Grade 3 [n=1]) were observed in 2 patients. The most frequent (N>1) drug related adverse events were Grade 1 or 2 dry skin, fatigue, nausea/vomiting and hot flushes. The possibility that dry skin may be a pharmacodynamic effect of BN83495 is being investigated in the ongoing protocol amendment. Dry skin was easily managed with hydrating/emollient creams. To date, the preliminary review of ECG data from the ongoing Phase I study suggest that BN83495 does not have an effect on ECG parameters. Four patients in the three higher dose groups have been reported to have stable disease for more than 6 months. A phase II European multicentre, randomised, open label study of oral STS inhibitor BN83495 versus megestrol acetate (MA) has been initiated in women with advanced or recurrent endometrial cancer. BN83495 is being used at a dose of 40 mg per day, which was determined to be the optimal biological dose in the phase 1 study described above. The primary objective is to determine the anti-tumour efficacy of BN83495 measured by progression free survival (PFS) at 6 months.

A phase I study has recently been initiated in castration resistant prostate cancer. The primary objective is to evaluate the pharmacodynamic profile of BN83495 at escalating doses (20, 40 and 60 mg) in patients with locally advanced or metastatic prostate cancer having prostate-specific antigen (PSA) progression on androgen ablative therapy. Both studies are ongoing and no data are currently available. BN83495 has not been studied in premenopausal females or in patients with renal or hepatic insufficiency. There has been no report of overdose with BN83495.

Dose Selection

The selected dose of BN83495 is an oral dose of 40 mg daily, given in the morning, fasted (30 minutes before breakfast). This is the recommended phase 2 dose determined from the phase 1 studies, which is currently being used in ongoing phase 2 studies.

Population to be Studied

Patients with histologically documented, unresectable, locally advanced, recurrent or metastatic (Stage IIIB or Stage IV) NSCLC who are otherwise eligible for treatment with an EGFR-TKI, or who are already receiving an EGFR-TKI.

Study Design

This is a pilot study conducted by the Lung Services at the Peter MacCallum Cancer Centre as an investigator-initiated trial. In stratum A, patients with NSCLC requiring treatment with an EGFR-TKI will be pre-treated with BN83495 (40 mg daily) for 3 days prior to starting the EGFR-TKI. In stratum B, patients already on an EGFR-TKI will receive BN83495 (40 mg daily). All patients will continue to receive BN83495 for a period of 12 weeks.

Treatment Assignment

This is a pilot study. Randomization is not required. Treatment assignment will be conducted following the final screening visit and based on the treatment history of the subject.

Number of Subjects

A pragmatic sample size of 20 patients with at least 5 in each stratum will be treated and observed. Any eligible patient who is registered but is withdrawn before receiving any study drug will be replaced.

Study Duration

A treatment period of 12 weeks with BN83495 is planned. For patients with no or minimal toxicity and minimal or no rash (in either strata) at 12 weeks, treatment with BN83495 will continue for an additional 3 months before being discontinued. If rash appears on discontinuation of treatment, BN83495 will be resumed for as long as the patient continues to receive the EGFR inhibitor. All patients will be followed up for 30 days after discontinuation of BN83495.

Selection and Withdrawal of Subjects

Inclusion Criteria
1. Patients with histologically documented, unresectable, locally advanced, recurrent or metastatic (Stage IIIB or Stage IV) NSCLC who are otherwise eligible for treatment with an EGFR inhibitor (erlotinib or gefitinib), or who are already receiving an EGFR inhibitor.
2. Patients who are scheduled to receive, or are already receiving a standard dose EGFR inhibitor.
3. The presence of any EGFR-TKI -related rash in patients already on treatment must be grade 2 or less.
4. Male or female patients aged 18 years or over who weigh 40 kg or more.
5. ECOG performance status of 0-2, inclusive.
6. Granulocyte count ≧1.5×109/L and platelet count >100×109/L.
7. Serum bilirubin must be ≦1.5× upper limit of normal (ULN). ALT must be ≦2× ULN.
8. Serum creatinine ≦1.5 ULN or creatinine clearance ≧60 ml/min.
9. Able to comply with study and follow-up procedures. Patients must be willing to be photographed.
10. a) Female patient of childbearing potential must have a negative pregnanacy test within one week prior to study entry OR have been amenorrhoeic for at least two years. b) All patients of reproductive potential must agree to use birth control for the duration of the study. This is only required for as long as the patient has reproductive potential. The type of birth control is a decision which should be made between the treating physician and the patient.
11. Patient has given written, informed consent to participate in the study.

Exclusion Criteria
1. Patients who cannot take oral medication.
2. Patients with prior prostate, breast or endometrial cancer unless in remission for
years.
3. Patients receiving other hormonally active agents.
4. Pregnancy or lactation.
5. Concurrent or recent history of significant skin disease, not related to EGFR inhibitor therapy.
6. Concurrent use of systemic or topical glucocorticoids (apart from intranasal and inhaled corticosteroids to treat rhinitis and asthma, respectively), for patients starting both an EGFR-TKI and BN83495. Patients already receiving an EGFR-TKI and topical glucocorticoids as part of their management are allowed.
7. Concurrent use of systemic carbonic anhydrase II inhibitors (e.g. acetazolamide, dichlorphenamide,methazolamide)
8. Serious illness or medical condition that precludes the safe administration of the trial treatment including, but not limited to, symptomatic congestive heart failure, unstable angina pectoris, cardiac arrhythmia, or psychiatric illness/social situations that would limit compliance with study requirements.
9. Patients unwilling or unable to comply with protocol and patients with a history of non-compliance or inability to grant informed consent.
10. Current participation in another clinical trial using an investigational agent.

Treatment of Subjects

EGFR Inhibitor Dosage, Administration, and Schedule

The oral dose of erlotinib is 150 mg daily, taken at least one hour before or two hours after the ingestion of food. The treatment will be via prescription and supplied through the PBS in Australia. Dose reductions are allowed as per standard care. This is within the specified treatment guidelines for erlotinib in Australia.

The oral dose of gefitinib is 250 mg daily, taken at least one hour before or two hours after the ingestion of food. The treatment will be via prescription and used in patients who can access the treatment via a Astra-Zeneca special access scheme as part of standard care. Dose reductions will be allowed as per standard care.

BN83495 Dosage, Administration, and Schedule

The oral dose of BN83495 is 40 mg daily, fasted (30 minutes before breakfast), given in the morning. BN83495 will be supplied by Ipsen Pty Ltd as tablets packed into conventional blister strips (10 tablets/blister). The 40 mg tablets are white oblong film-coated tablets of approximately 15 mm length and 7 mm width. Records of study medication used, dosages administered, and intervals between visits will be kept during the study by the site staff or delegated to the pharmacy personnel. BN83495 and erlotinib accountability will be logged by study/pharmacy staff for the duration of the study. Patients will be asked to return all unused clinical trial stock of BN83495 tablets at the discontinuation of treatment.

### BIBLIOGRAPHY

Agero A L, Dusza S W, Benvenuto-Andrade C, et al. Dermatologic side effects associated with the epidermal growth factor receptor inhibitors. J Am Acad Dermatol 2006; 55:657-670.
Basti S. Ocular toxicities of epidermal growth factor receptor inhibitors and their management. Cancer Nurs 2007; 30(4 Suppl 1):S10-16.
Boone S L, Rademaker A, Liu D, et al: Impact and management of skin toxicity associated with anti-epidermal growth factor receptor therapy: Survey results. Oncology 72:152-159, 2007.
Burtness B, Anadkat M, Basti S et al. NCCN Task Force Report: Management of dermatologic and other toxicities associated with EGFR inhibition in patients with cancer. Journal of the National Comprehensive Cancer Network. 2009 May; 7 Suppl 1:S5-S21; quiz S22-4.
Castillo L, Etienne-Grimaldi M C, Fischel J L, et al. Pharmacological background of EGFR targeting. Ann Oncol 2004; 15:1007-1012.
Cunningham D, Humblet Y, Siena S, et al. Cetuximab monotherapy and cetuximab plus irinotecan in irinotecan-refractory metastatic colorectal cancer. N Engl J Med 2004; 351:337-345.
Fischer D S, Chander S K, Woo L W L et al. Novel D-ring modified steroid derivatives as potent, non-estrogenic steroid sulfatase inhibitors with in vivo activity. J Steroid Biochem Mol Biol 2003; 84:343-349.
Foster P A, Newman S P, Chander S K et al. In vivo efficacy of STX213, a second-generation steroid sulfatase inhibitor, for hormone-dependent breast cancer therapy. Clin Cancer Res 2006, 12:5543-5549.
Gridelli C, Maione P, Amoroso D, et al. Clinical significance and treatment of skin rash from erlotinib in non-small cell lung cancer patients: results of an Experts Panel Meeting. Crit Rev Oncol Hematol 2008; 66:155-162.
Herbst R S, Maddox A M, Rothenberg M L, et al. Selective oral epidermal growth factor receptor tyrosine kinase inhibitor ZD1839 is generally well-tolerated and has activity in non-small-cell lung cancer and other solid tumours: results of a phase I trial. J Clin Oncol 2002; 20:3815-3825.
International Patent Application No. PCT/GB92/01587
International Union of Biochemistry and Molecular Biology, <http://www.chem.qmul.ac.uK/iubmb/enzyme/EC3/1/6/2.html>.
Jemal A, Siegel R, Ward E, Hao Y, Xu J and Thun M J. Cancer Statistics, 2009. CA Cancer J Clin 2009, 59; 225-249.
Melosky B, Burkes R, Rayson D et al., Management of skin rash during EGFR-targeted monoclonal antibody treatment for gastro intestinal malignancies: Canadian recommendations. Current Oncology 2009, 16(1): 16-26.
Mendelsohn J. Targeting the epidermal growth factor receptor for cancer therapy. J Clin Oncol 2002, 20:1s-13s
National Cancer Institute Common Terminology Criteria for Adverse Events (CTCAE) version 3.0. Available from: (http://ctep.cancer.gov/protocolDevelopment/electronic_applications/docs/ctcaev3.pdf).
Purohit A, Woo L W L, Potter B V L et al. In vivo inhibition of estrone sulfatase activity and growth of nitrosomethylurea-induced mammary tumours by 667 COUMATE. Cancer Res 2000; 60:3394-3396
Reed M J, Purohit A, Woo L W L et al. Steroid Sulfatase: Molecular Biology, Regulation, and Inhibition. Endocrine Reviews 2005, 26 (2): 171-202
Remington's Pharmaceutical Sciences, 20th Ed. Williams and Wilkins, 2000
Roche Products—Tarceva (erlotinib) Product Information. 24 Mar. 2009
US Patent Application Publication No. US 2009/0182000 A1
U.S. Pat. No. 5,616,574
Weber R S, Lustig R, Glisson B, et al. A phase II trial of ZD 1869 for advanced cutaneous squamous cell carcinoma of the head and neck [Abstract]. J Clin Oncol 2007, 25(Suppl 1):Abstract 6038.

1. A method of treating and/or preventing a toxicity associated with epidermal growth factor receptor (EGFR) inhibitor therapy in a subject, the method comprising administering to the subject an effective amount of a steroid sulfatase (STS) inhibitor.

2. The method of claim 1, wherein the subject is undergoing EGFR inhibitor therapy for the treatment of cancer.

3. The method according to claim 2 further comprising administering to the subject an effective amount of an aromatase inhibitor.

4. The method according to claim 1, wherein the toxicity is selected from the group consisting of: ocular toxicity; and dermatologic toxicity.

5. The method according to claim 4, wherein the toxicity is dermatologic toxicity.

6. The method according to claim 5, wherein the dermatologic toxicity is papulopustular rash.

7. The method according to claim 1, wherein the EGFR inhibitor is selected from the group consisting of: a small molecule; an antibody or derivative or fragment thereof; and any combination thereof.

8. The method according to claim 7, wherein the small molecule is a tyrosine kinase inhibitor selected from the group consisting of erlotinib; gefitinib; lapatinib; and any combination thereof

9. The method according to claim 7, wherein the antibody is selected from the group consisting of: cetuximab; panitumumab; and any combination thereof.

10. The method according to claim 1, wherein the STS inhibitor is selected from the group consisting of: alternative STS substrates; reversible STS inhibitors; irreversible STS inhibitors; and any combination thereof.

11. The method according to claim 10, wherein the STS inhibitor is the irreversible nonsteroidal STS inhibitor STX64.

12. The method according to claims 3, wherein the aromatase inhibitor is selected from the group consisting of: anastrazole; exemestane; letrozole; and any combination thereof.

13. The method according to claim 2, wherein the cancer comprises cells that express wildtype k-ras and/or wildtype b-raf.

14. The method according to claim 2, wherein the cancer is hormone-dependent.

15. The method according to claim 2, wherein the cancer is selected from the group consisting of: advanced colorectal cancer; operable-early colorectal cancer; head and neck cancer; pancreatic cancer; non-small cell lung cancer; breast cancer; gastro-intestinal cancer; colon cancer; skin cancer; other solid tumours; leukemia; and lymphoma.

16. A pharmaceutical composition comprising an STS inhibitor for the treatment and/or prevention of a toxicity associated with EGFR inhibitor therapy in a subject, an EGFR inhibitor and/or an aromatase inhibitor and a pharmaceutically-acceptable carrier.

17. A kit comprising an STS inhibitor for the treatment and/or prevention of a toxicity associated with EGFR inhibitor therapy in a subject, an EGFR inhibitor and/or an aromatase inhibitor and a pharmaceutically-acceptable carrier.

18. A method of treatment of cancer comprising administering to a subject in need thereof a therapeutically effective amount of an EGFR inhibitor and a therapeutically effective amount of a STS inhibitor.

19. A method of treating and/or preventing a papulopustular rash side effect associated with epidermal growth factor receptor (EGFR) inhibitor therapy in a subject being treated with EGFR inhibitor therapy for cancer, the method comprising administering to the subject an effective amount of the irreversible nonsteroidal STS inhibitor STX64.
